(19) United States
(12) Patent Application Publication
(10) Pub. No.: US 2010/0030116 A1
(43) Pub. Date: Feb. 4, 2010

(54) TARGETING DEVICE

(76) Inventor: Gursharan Singh Chana, West Midlands (GB)

Correspondence Address: ICE MILLER LLP, ONE AMERICAN SQUARE, SUITE 3100, INDIANAPOLIS, IN, 46282-0200, US

(21) Appl. No.: 12/441,933

(22) PCT Filed: Sep. 19, 2007
(86) PCT No.: PCT/GB2007/003535

§ 371 Date: Aug. 25, 2009

Related U.S. Application Data
(63) Continuation of application No. 11/535,051, filed on Sep. 25, 2006, now abandoned.

(30) Foreign Application Priority Data
Sep. 19, 2006 (GB) 0618359.4

(51) Int. Cl. A61B 5/103; A61B 17/58

(52) U.S. Cl. 600/587; 606/96

(57) ABSTRACT

A targeting device (20) for use in hip surgery to allow the position of the centreline of the femoral neck to be located, the device comprising: (a) one or more location sensor device (22a, 22b, 23a, 23b), wherein at least part of each location sensor device is movable to a position inside the body that is over the femoral neck, the device or devices being suitable for obtaining data relating to the location of the surface of the femoral neck with respect to a reference position, in both the anterior-posterior and superior-inferior planes; (b) a guide sleeve (32), having a bore capable of receiving a guide wire; and (c) a computer device with software for receiving data from the location sensor device or devices and processing this data so as to determine the location of the centreline of the femoral neck; wherein the guide sleeve can be moved in the anterior, posterior, medial and lateral directions, so that it can be moved so as to be in line with the centreline of the femoral neck as determined by the computer device.

The present invention relates to a targeting device, particularly for use in hip surgery and more particularly for use in hip replacement surgery or hip resurfacing surgery. The invention also relates to the use of the device in surgery, particularly minimally invasive surgery.

When performing hip replacement surgery it is essential to be able to accurately determine where the centre of the base of the femoral neck and the centreline through the femoral neck lie to allow correct reaming fitting of the replacement head.

Equally, when performing hip resurfacing surgery it is essential to be able to accurately determine where the centreline through the femoral neck and femoral head lies to allow correct reaming of the femoral head and correct fitting of the prosthesis onto the femoral head.

In the event that the centreline is not accurately determined, there is a high risk of femoral neck fracture and failure of the implant, thus leading to a need for revision surgery. As well as being a secondary operation this can be quite distressing for the patient and is also a more expensive procedure.

The position of the femoral head does not always assist in determining the position of the centre of the femoral neck and the centreline through the femoral neck as the head may not be centrally positioned in relation to the femoral neck due to deformity of the femoral head.

Accordingly there is the need for a targeting device which is suitable to allow the position of the centre of the femoral neck and/or the centreline through the femoral neck and femoral head (hereinafter reference to “the centreline of the femoral neck” is used to cover both of these references) to be determined, which device can be used in minimally invasive surgical methods and in conventional open surgical methods.

In a first aspect of the invention, there is provided a targeting device for use in hip surgery to allow the position of the centreline of the femoral neck to be located, the device comprising:
one or more location sensor device, wherein at least part of each location sensor device is movable to a position inside the body that is over the femoral neck, the device or devices being suitable for obtaining data relating to the location of the surface of the femoral neck with respect to a reference position, in both the anterior-posterior and superior-inferior planes;
a guide sleeve, having a bore capable of receiving a guide wire; and
a computer device with software for receiving data from the location sensor device or devices and processing this data so as to determine the location of the centreline of the femoral neck;
   wherein the guide sleeve can be moved in the anterior, posterior, medial and lateral directions, so that it can be moved so as to be in line with the centreline of the femoral neck as determined by the computer device.

In one embodiment, part of each sensor device is movable so as to be in contact with the femoral neck surface, wherein each sensor device includes a sensor for obtaining information about the location of this part that is movable so as to be in contact with the femoral neck surface, such that the device or devices are suitable for obtaining data relating to the location of the surface of the femoral neck with respect to a reference position, in both the anterior-posterior and superior-inferior planes.

Accordingly, in one embodiment of the first aspect of the invention, there is provided a targeting device for use in hip surgery to allow the position of the centreline of the femoral neck to be located, the device comprising:
one or more location sensor device, wherein at least part of each location sensor device is movable to a position inside the body that is over the femoral neck and wherein part of each sensor device is movable so as to be in contact with the femoral neck surface, wherein each sensor device includes a sensor for obtaining information about the location of this part that is movable so as to be in contact with the femoral neck surface, such that the device or devices are suitable for obtaining data relating to the location of the surface of the femoral neck with respect to a reference position, in both the anterior-posterior and superior-inferior planes;
a guide sleeve, having a bore capable of receiving a guide wire; and
a computer device with software for receiving data from the location sensor device or devices and processing this data so as to determine the location of the centreline of the femoral neck,
   wherein the guide sleeve can be moved in the anterior, posterior, medial and lateral directions, so that it can be moved so as to be in line with the centreline of the femoral neck as determined by the computer device.

Preferably, the targeting device includes a body portion that extends from a distal end to a proximal end. Preferably, the guide sleeve is part of the body portion and the body portion is movable so as to cause corresponding movement of the guide sleeve, so that the guide sleeve may be moved in the anterior, posterior, medial and lateral directions, so that it can be moved so as to be in line with the centreline of the femoral neck as determined by the computer device.

The guide sleeve preferably extends from the distal end to the proximal end of the body portion. In one embodiment, the guide sleeve is substantially aligned with the central elongate axis of the body portion.

In one embodiment, the body portion is substantially cylindrical in shape.

The body portion suitably is provided with the location sensor device(s). Preferably, at least part of the location sensor device(s) is located within the body portion, and at least part of the location sensor device(s) extends from the proximal end of the body portion.

In one embodiment, the body portion is separate from the computer device.

The body portion may suitably have a length from its proximal end to its distal end of from 50 to 250 mm, such as from 60 to 200 mm, e.g. from 75 to 125 mm.

The body portion may suitably have a width of up to 150 mm, preferably up to 120 mm, more preferably up to 100 mm, such as from 50 to 100 mm, e.g. from 60 to 95 mm, such as from 75 to 90 mm.

Preferably there are two or more location sensor devices, with at least one device being able to obtain data in the anterior-posterior plane and at least one device being able to obtain data in the superior-inferior plane. There may be three or more location sensor devices, preferably four or more location sensor devices. Preferably, there are at least two devices being able to obtain data in the anterior-posterior plane and at least two devices being able to obtain data in the superior-inferior plane.

Preferably, there are three or more angularly spaced location sensor devices, more preferably four or more angularly spaced location sensor devices. For example, there may be four angularly spaced location sensor devices, spaced at angles of 90° apart.

In a preferred embodiment, there are at least two sets of location sensor devices, wherein each set includes one or more location sensor device, wherein at least part of each location sensor device is movable to a position inside the body that is over the femoral neck and wherein part of each sensor device is movable so as to be in contact with the femoral neck surface, wherein each sensor device includes a sensor for obtaining information about the location of the part that is movable so as to be in contact with the femoral neck surface, such that each set of location sensor devices is suitable for obtaining data relating to the location of the surface of the femoral neck with respect to a reference position, in both the anterior-posterior and superior-inferior planes.

In one preferred embodiment, there are exactly two sets of location sensor devices. In another embodiment, there are more than two sets of location sensor devices. In one embodiment, there is only one set of location devices.

The sets of location sensor devices are spaced apart such that they will contact the femoral neck surface at different distances from the femoral head. For example, the sets of location sensor devices may be spaced apart by a distance of 5 mm or more, such as from 5 mm to 40 mm; preferably 7 mm or more, more preferably 10 mm or more, for example from 10 mm to 35 mm; such as 12 mm or more, 15 mm or more, 20 mm or more; for example from 20 mm to 30 mm.

Preferably, each set of location sensor devices comprises two or more location sensor devices, with at least one device in each set being able to obtain data in the anterior-posterior plane and at least one device in each set being able to obtain data in the superior-inferior plane. Each set of location sensor devices may comprise three or more location sensor devices. Preferably each set of location sensor devices comprises four or more location sensor devices. Preferably, each set of location sensor devices has at least two devices being able to obtain data in the anterior-posterior plane and at least two devices being able to obtain data in the superior-inferior plane.

Preferably, each set of location sensor devices comprises three or more angularly spaced location sensor devices, more preferably four or more angularly spaced location sensor devices. For example, each set may have four angularly spaced location sensor devices, spaced at angles of 90° apart.

In one embodiment, most or all of each location sensor device is movable to a position inside the body. In an alternative embodiment, only the part of each sensor device that is movable so as to be in contact with the femoral neck surface is movable to a position inside the body.

Preferably the location sensor devices are each electro-mechanical devices.

The part of each sensor device that is movable so as to be in contact with the femoral neck surface may suitably be a lever. The lever has one fixed end and one movable end and pivots about its fixed end. The movable end of the lever moves from a first, retracted, position, to a second, engaging, position. In use, the first, retracted, position will be where the movable end of the lever is not contacting the femoral neck surface, and the second, engaging, position will be where it is contacting the femoral neck surface.

In a preferred embodiment, the lever is biased towards the second position.

In a preferred embodiment, movement of the lever from the first position to the second position is caused by a biasing means, such as a spring or other elastic device that exerts a biasing force. In one embodiment, the movement of the lever from the first position to the second position is caused by a torsion spring.

Preferably, the movement of each lever is limited by a stopper; for example a stopper pin. The stopper pin(s) may be mounted on the body portion of the targeting device.

Preferably, each lever is mounted at its fixed end on an elongate shaft that extends from a distal end to a proximal end; preferably at or near the proximal end of the shaft. Each lever is mounted on the shaft such that movement of the lever causes a corresponding movement of the shaft.

The lever may move in a plane perpendicular to the elongate shaft (“rotating lever”) or the lever may move in a plane parallel to the elongate shaft (“standard lever”). Preferably the lever is a rotating lever.

Accordingly, in a preferred embodiment the lever moves from a first position to a second position in an arc in a plane that is perpendicular to the length of the elongate shaft.

The distal end of each shaft may be mounted on the body portion of the targeting device. Preferably, the shaft passes through the proximal end of the body portion and the distal end of each shaft is mounted inside the body portion of the targeting device.

Each shaft is moveably mounted on the body portion, such that the shaft moves with the movement of the lever. For example, when the lever is a rotating lever, the shaft rotates with the movement of the lever.

Accordingly, in one embodiment the shaft may be supported on the body portion using movement-permitting means, such as ball bearings or rollers.

The shafts may have any suitable length but preferably the length of the shaft from the proximal end of the body portion to the proximal end of the shaft is from 30 to 150 mm, such as from 40 to 120 mm, e.g. from 50 to 100 mm, such as from 60 to 80 mm.

Preferably, the lever can be moved from the second position, towards which it is biased, towards the first position, by a manual location adjustor. This may comprise a wheel, which can be rotated by the user, to counteract the force of the biasing means. Preferably, the wheel is provided with a handle that is turned by the user in order to rotate the wheel.

For example, there may be an arm extending from each of the shafts, and the wheel may be provided with pins that abut each of these arms. The turning of the wheel will move the pins, which will then push the arm(s) and this will in turn move the shafts, which will correspondingly move the levers towards their first position.

Such a manual location adjustor can be used to move the levers out of the way to facilitate movement of the levers over the femoral head to reach the desired location over the femoral neck.

The device may be also provided with a securing system, which enables the user to secure the levers in the first position. This ensures that the levers of the targeting device can be readily placed over, or removed from, the femoral neck, without needing to continually activate the manual location adjustor.

The device may be also provided with a securing system, which enables the user to secure the levers in the second position. This ensures that the targeting device can locked onto the femoral neck once the guide sleeve has been moved so as to be in line with the centreline of the femoral neck as determined by the computer device, to keep it in the correct position.

When the part of each sensor device that is movable so as to be in contact with the femoral neck surface is a standard lever, the sensor for obtaining information about the location of this lever may be a linear transducer.

When the sensor for obtaining information about the location of the part that is movable so as to be in contact with the femoral neck surface is a rotating lever, the sensor for obtaining information about the location of this lever may be a rotary encoder, such as a rotary magnetic encoder or a rotary optical encoder, or a potentiometer.

In a preferred embodiment, the part of each location sensor device that is movable so as to be in contact with the femoral neck surface is a rotating lever and the sensor for obtaining information about the location of this lever is a rotary shaft encoder, such as a magnetic rotary shaft encoder, for example an absolute magnetic rotary shaft encoder.

The sensor for obtaining information about the location of the part that is movable so as to be in contact with the femoral neck surface is suitably a miniature device, for example with a diameter of 20 mm or less, such as 15 mm or less, e.g. 12.5 mm or less.

The sensor for obtaining information about the location of the part that is movable so as to be in contact with the femoral neck surface is suitably able to withstand temperatures of 100° C. or higher, such as 110° C. or higher, e.g. from 115° C. to 125° C. or higher; preferably from 120° C. to 125° C. or higher, for example from 125° C. to 135° C. or higher.

Examples of sensors that may be used include magnetic encoders available from US Digital, such as the MA3 miniature absolute magnetic shaft encoders.

The sensor may suitably obtain information about the movement of the lever from the corresponding movement of the shaft.

In a preferred embodiment, the lever is a rotating lever mounted on the end of a shaft and the corresponding rotation of the shaft is measured by the sensor (such as a rotary encoder or a potentiometer). Preferably, the sensor is connected to the shaft via a coupling bush, which transmits the corresponding rotation of the shaft to the sensor.

The sensor is held in a fixed position relative to the body portion.

In one embodiment, the location sensor devices comprise one or more rotating levers and a corresponding number of sensory devices, which are rotary encoders or potentiometers. The or each lever may be arranged so as to be able to move from a neutral position, located radially with respect to the femoral neck, to a position abutting the surface of the femoral neck. The or each sensor device is arranged to be able to record the distance moved by the lever from the neutral position to the position abutting the surface of the femoral neck by measurement of the angle of rotation of the lever. Therefore the location of the point of the surface of the femoral neck abutted by the lever may be calculated.

The guide sleeve may be any suitable shape and size for receiving a guide wire in its bore. Suitably, the guide sleeve may be an elongate cylindrical shape with a bore suitable for receiving a guide wire. The guide sleeve may in particular be a conventional guide sleeve for a guide wire as known in the art.

The software may be any software capable of receiving and processing the data from the or each sensor device, so as to determine the position of the femoral neck centreline. Clearly, the exact nature of the software will vary depending upon the sensor device used; the software must be able to process the data recorded by the sensor device and from this determine the position of the centreline of the femoral neck.

In a preferred embodiment, the targeting device further comprises a screen and the software is such that, having determined the location of the centreline of the femoral neck, it provides information regarding the centreline location on the screen so that a person can manually move the guide sleeve in line with this calculated centreline position.

Preferably the location sensor device(s) provide information about the location of the surface of the femoral neck as the targeting device is moved, which data is passed to the computer device. This data can be processed and the screen can therefore be updated with up to date information.

In one embodiment, the targeting device comprises a screen and the software is such that, having determined the location of the centreline of the femoral neck, it provides an image of the centreline location on the screen so that a person can manually move the guide sleeve in line with this calculated centreline position.

In one embodiment, the targeting device further comprises a screen and there is at least one set of location sensor devices, which provide information about the location of the surface of the femoral neck. Each set of information about the location of the surface of the femoral neck may be presented on the screen, as an ellipse that represents an estimate of the location of the surface of the femoral neck. The centre of this ellipse is preferably also shown. The location of the centreline of the guide sleeve is suitably also be presented on the screen. Accordingly, a person can manually move the guide sleeve until the location of the centreline of the guide sleeve is lined up with the centre of the ellipse.

Preferably the location sensor devices in each set are such that they provide at least four data points in respect of the location of the surface of the femoral neck, which data points are used to determine the ellipse.

Preferably the location sensor device(s) provide information about the location of the surface of the femoral neck as the targeting device is moved, which data is passed to the computer device. This data can be processed and the screen can therefore be updated with up to date information regarding the ellipse that represents an estimate of the location of the surface of the femoral neck and the location of the centreline of the guide sleeve.

In one embodiment, an indication may be given on the screen when the centreline of the guide sleeve has been lined up with the calculated centreline position of the femoral neck.

Preferably, there are at least two sets of location sensor devices, which provide information about the location of the surface of the femoral neck from two different positions on the femoral neck. Each of these two or more sets of information about the location of the surface of the femoral neck may be presented on the screen, as two or more ellipses. The centre of each ellipse is preferably also shown. The location of the centreline of the guide sleeve is suitably also be presented on the screen. Accordingly, a person can manually move the guide sleeve until the location of the centreline of the guide sleeve is lined up with the centre of the ellipses.

The use of two or more sets two sets of location sensor devices, which provide information about the location of the surface of the femoral neck from two different positions on the femoral neck, improves the accuracy of the calculated centreline position.

In an alternative embodiment of the first aspect of the invention, there is provided a targeting device for use in hip surgery to allow the position of the centreline of the femoral neck to be located, the device comprising:
one or more location sensor device, movable to a position inside the body that is over the femoral neck and radially spaced therefrom, the device or devices being suitable for obtaining data relating to the location of the surface of the femoral neck with respect to a reference position, in both the anterior-posterior and superior-inferior planes;
a guide sleeve, having a bore capable of receiving a guide wire;
a guide sleeve location adjustor, suitable for moving the guide sleeve in the anterior, posterior, medial and lateral directions; and
a computer device with software for receiving data from the sensor device or devices and processing this data so as to determine the location of the centreline of the femoral neck.

The guide sleeve location adjustor permits the guide sleeve to be moved, so as to be in line with the centreline of the femoral neck as determined by the computer device.

The one or more sensor devices may be any device or combination of devices that permits direct or indirect measurement of distance, in both the anterior-posterior and superior-inferior planes, between a reference location and the surface of the femoral neck when the one or more sensor devices are located at a position or positions inside the body, over the femoral neck and radially spaced therefrom.

In one embodiment, there may be one or more sensor devices that are movable so as to obtain data at two or more different radial locations around the femoral neck, with at least one location being such that data is obtained in the anterior-posterior plane and at least one location being such that data is obtained in the superior-inferior plane.

Preferably, the one or more sensor devices are movable so as to obtain data at three or more different radial locations around the femoral neck, more preferably four or more different radial locations around the femoral neck, for example six or more different radial locations around the femoral neck.

Preferably, the one or more sensor devices are movable to two or more different radial locations where data is obtained in the superior-inferior plane, such as three or more different radial locations where data is obtained in the superior-inferior plane, for example four or more different radial locations where data is obtained in the superior-inferior plane.

Preferably, the one or more sensor devices are movable to two or more different radial locations where data is obtained in the anterior-posterior plane, such as three or more different radial locations where data is obtained in the anterior-posterior plane, for example four or more different radial locations where data is obtained in the anterior-posterior plane.

In another embodiment, there may be two or more angularly spaced sensor devices, which may be fixed or movable, with at least one device being able to obtain data in the anterior-posterior plane and at least one device being able to obtain data in the superior-inferior plane.

Preferably, there are three or more angularly spaced sensor devices, more preferably four or more angularly spaced sensor devices, for example six or more angularly spaced sensor devices.

For example, there may be four angularly spaced sensor devices, spaced at angles of 90° apart.

Preferably, there are two or more devices able to obtain data in the superior-inferior plane, such as three or more devices able to obtain data in the superior-inferior plane, for example four or more devices able to obtain data in the superior-inferior plane.

Preferably, there are two or more devices able to obtain data in the anterior-posterior plane, such as three or more devices able to obtain data in the anterior-posterior plane, for example four or more devices able to obtain data in the anterior-posterior plane.

The sensor devices may suitably be selected from electro-mechanical devices, radar devices, infra-red devices, ultrasound devices and electro-magnetic devices.

Electro-mechanical devices can be used to obtain data relating to the distance between a reference location and the surface of the femoral neck. Specifically, the or each electromechanical device may be arranged so as to be able to move from a neutral position, located radially with respect to the femoral neck, to a position abutting the surface of the femoral neck. The or each electromechanical device is arranged to be able to record the distance moved by the electromechanical device from the neutral position to the position abutting the surface of the femoral neck. Accordingly, the reference location is the neutral position of the electromechanical device.

Preferably, the or each electromechanical device is be able to move in a straight line from the neutral position to the position abutting the surface of the femoral neck.

In particular, the or each sensor device may comprise an electronically driven motor that is be able to move in a straight line from the neutral position to the position abutting the surface of the femoral neck.

Preferably, the or each electromechanical device is also able to move back from the position abutting the surface of the femoral neck to the neutral position. The distance travelled back from the position abutting the surface of the femoral neck to the neutral position may also be recordable by the or each sensor device.

Radar devices can be used to obtain data relating to the distance between a reference location and the surface of the femoral neck. Specifically, the or each radar device may be arranged so as to be able to emit a radar beam from a radial position with respect to the femoral neck, towards the femoral neck. The radar beam will reach the femoral neck, bounce off the neck and arrive back at the radar device. The or each radar device is arranged to be able to record the time taken for the beam to arrive back at the radar device. Accordingly, the reference location is the radial position of the radar device with respect to the femoral neck.

From the information relating to how long the radar beam took to travel a distance equal to two times the distance from the reference location to the femoral neck, the distance between the reference location and the surface of the femoral neck can be established.

Infra-red devices can be used to obtain data relating to the distance between a reference location and the surface of the femoral neck. Specifically, the or each infra-red device may be arranged so as to be able to emit an infra-red beam from a radial position with respect to the femoral neck, towards the femoral neck. The infra-red beam will reach the femoral neck, bounce off the neck and arrive back at the infra-red device. The or each infra-red device is arranged to be able to record the time taken for the beam to arrive back at the infra-red device. Accordingly, the reference location is the radial position of the infra-red device with respect to the femoral neck.

From the information relating to how long the infra-red beam took to travel a distance equal to two times the distance from the reference location to the femoral neck, the distance between the reference location and the surface of the femoral neck can be established.

Ultrasound devices can be used to obtain data relating to the distance between a reference location and the surface of the femoral neck. Specifically, the or each ultrasound device may be arranged so as to be able to emit an ultrasound wave from a radial position with respect to the femoral neck, towards the femoral neck. The ultrasound wave will reach the femoral neck, bounce off the neck and arrive back at the ultrasound device. The or each ultrasound device is arranged to be able to record the time taken for the ultrasound wave to arrive back at the ultrasound device. Accordingly, the reference location is the radial position of the ultrasound device with respect to the femoral neck.

From the information relating to how long the ultrasound wave took to travel a distance equal to two times the distance from the reference location to the femoral neck, the distance between the reference location and the surface of the femoral neck can be established.

Electro-magnetic devices can be used to obtain data relating to the distance between a reference location and the surface of the femoral neck. Specifically, the or each electro-magnetic device may be arranged so as to be able to emit an electro-magnetic wave from a radial position with respect to the femoral neck, towards the femoral neck. The electro-magnetic wave will reach the femoral neck, bounce off the neck and arrive back at the electromagnetic device. The or each electro-magnetic device is arranged to be able to record the time taken for the electro-magnetic wave to arrive back at the electromagnetic device. Accordingly, the reference location is the radial position of the electro-magnetic device with respect to the femoral neck.

From the information relating to how long the electro-magnetic wave took to travel a distance equal to two times the distance from the reference location to the femoral neck, the distance between the reference location and the surface of the femoral neck can be established.

Clearly, any other suitable sensor device could also be used.

In one embodiment, the targeting device comprises:
one or more retractable arm, each of which can move from a retracted state to an extended state.

There may be two or more retractable arms, for example three or more, such as four or more retractable arms. The retractable arms may be extended and retracted by any suitable system, for example the retractable arms may be movable by an electrometrical system. In particular, this system may comprise combinations of known motors and electronic devices that permit the arms to be extended and retracted.

The extension and retraction of the arms may be controlled by a person.

In one embodiment, the retractable arms are connected to the computer device and software, and the extension and retraction of the arms is controlled by the software.

The one or more sensor devices may be located on the one or more retractable arms. In particular, the sensor device or devices may each be located on the distal end of a retractable arm. Each retractable arm may be provided with one or more sensor device. Preferably, there are the same number of arms as sensor devices, and a single sensor device is provided on each arm.

Movement of the one or more retractable arms from the retracted state to the extended state can move the one or more sensor devices from a position outside the body to a position inside the body, over the femoral neck and radially spaced therefrom.

The one or more retractable arms may be any suitable size and shape. The one or more retractable arms may be a curved shape, e.g. an arc shape. This allows the one or more sensor devices to be readily moved over the femoral head, when present, and to a position located over the femoral neck.

The one or more retractable arms may suitably be rigid, or they may be flexible.

The targeting device may further comprise a body portion, on which the retractable arms may be mounted. Preferably, the retractable arms are mounted at one end of the body portion.

The use of one or more retractable arms, with the sensor devices provided on the arms, is advantageous as it minimises the amount of equipment that needs to be placed inside the body. In particular, only the arms and sensor devices need enter the body; the remainder of the device can remain outside the body throughout the procedure.

In one embodiment, the targeting device comprises:
a sleeve component, comprising a first wall having an exterior surface and an interior surface that defines an inner bore, the wall running from a proximal end of the sleeve component to a distal end of the sleeve component, the sleeve component being shaped and sized such that it can be placed over the femoral neck with some or all of the femoral neck located within the bore.

The one or more sensor devices can be located in the sleeve component. The sleeve component can be moved from a position outside the body, to a position inside the body, with some or all of the femoral neck located within the bore of the sleeve component. The one or more sensor devices can thus be moved from a position outside the body to a position inside the body, over the femoral neck and radially spaced therefrom.

The sleeve component is shaped and sized such that it can be placed over the femoral neck with some or all of the femoral neck located within its bore.

The first wall of the sleeve component may form a cylinder shape. Alternatively, the first wall of the sleeve component may form a hollow square prism shape, a hollow rectangular prism shape, a hollow hexagonal prism shape, or a hollow octagonal prism shape. The first wall of the sleeve component could also be any other regular or irregular shape, provide that it defines a bore within which some or all of the femoral neck can be located.

Suitably, the bore of the sleeve component has a diameter of 2.5 cm or more, such as from 3 cm to 8 cm, preferably from 3 cm to 7 cm, for example from 3.5 cm to 6.5 cm, more preferably from 4 cm to 6.5 cm.

The sleeve component preferably has a length such that it can be placed over the femoral head down to the base of the femoral neck. The sleeve component may suitably have a length of 3 cm or more, such as from 3.5 cm to 7 cm, preferably from 4 cm to 6 cm.

In one embodiment, the sleeve component is provided with a second wall, with the second wall being located outside of the first wall and being shaped so as to define a cavity between the first wall and the second wall.

In one embodiment, the second wall is of a substantially corresponding shape to the first wall, so that the cavity between the first and second wall is of a substantially constant depth. In an alternative embodiment, the second wall is of a different shape to the first wall.

The cavity formed between the first and second walls may be any suitable depth. Preferably, the cavity is large enough that it can house some or all of the sensor devices. However, it is clearly preferred that the depth of the cavity, and hence the overall diameter of the device, is kept as small as possible whilst achieving the desired function, in order to make the device more readily usable during surgery. Accordingly, it is preferred that the depth of the cavity between the first and second walls is 2 cm or less, more preferably 1.5 cm or less, most preferably 1 cm or less.

The distal end of the sleeve component is open so that it can be placed over the femoral head/femoral neck. The proximal end of the sleeve component may be open or may be closed apart from the guide sleeve holder.

The guide sleeve may be any suitable shape and size for receiving a guide wire in its bore. Suitably, the guide sleeve may be an elongate cylindrical shape with a bore suitable for receiving a guide wire. The guide sleeve may in particular be a conventional guide sleeve for a guide wire as known in the art.

The guide sleeve location adjustor may be electrometrical. In particular, it may comprise combinations of known motors and electronic devices that permit the guide sleeve to be moved in the anterior, posterior, medial and lateral directions.

In one embodiment, the guide sleeve location adjustor is connected to the computer device and software, and the movement of the guide sleeve is controlled by the software.

In an alternative embodiment, the guide sleeve location adjustor is controlled by a person.

The software may be any software capable of receiving and processing the data from the or each sensor device, so as to determine the position of the femoral neck centreline. Clearly, the exact nature of the software will vary depending upon the sensor device used; the software must be able to process the data recorded by the sensor device and from this determine the position of the centreline of the femoral neck.

In one embodiment, the software is such that, having determined the location of the centreline of the femoral neck, it controls the guide sleeve location adjustor and causes the adjustor to move the guide sleeve in line with this calculated centreline position.

In an alternative embodiment, the targeting device further comprises a screen and the software is such that, having determined the location of the centreline of the femoral neck, it provides an image of the femoral neck and its centreline location on the screen so that a person can manually use the guide sleeve location adjustor to move the guide sleeve in line with this calculated centreline position.

Clearly, in all embodiments of the first aspect, the targeting device can be made from any suitable material, such as metal or plastics material. Those portions of the device that are intended to enter into the body should be made from surgically acceptable material.

Preferably the portions of the device that are intended to enter into the body should be made from material that can be steam sterilized, for example at a pressure of from 1 to 3 bar, such as 1.5 to 2.5 bar, e.g. about 2 bar, and at a temperature of from 120 to 140° C., such as from 125 to 135° C.

In a second aspect of the invention, there is provided a method of locating the centreline of the osteotomised base of the femoral neck at the head-neck junction, which method comprises:
providing a targeting device in accordance with the first aspect;
moving at least part of the or each location sensor device to a position inside the body, over the femoral neck;
using the or each location sensor device to obtain data relating to the location of the surface of the femoral neck with respect to a reference position in at least one location in the anterior-posterior plane and at least one location in the superior-inferior plane;
using the software to determine the position of the centreline of the femoral neck based on the data received from the or each location sensor device;
moving the guide sleeve to be in line with the determined centreline.

Preferably the method further comprises the step of:
passing a guide wire through the guide sleeve to mark the centreline of the osteotomised femoral neck.

In a third aspect of the invention, there is provided a method of locating the centreline through the femoral neck and femoral head, which method comprises:
providing a targeting device in accordance with the first aspect;
moving at least part of the or each location sensor device to a position inside the body, over the femoral neck;
using the or each location sensor device to obtain data relating to the location of the surface of the femoral neck with respect to a reference position in at least one location in the anterior-posterior plane and at least one location in the superior-inferior plane;
using the software to determine the position of the centreline of the femoral head and neck based on the data received from the or each sensor device;
moving the guide sleeve to be in line with the determined centreline.

Preferably the method further comprises the step of:
passing a guide wire through the guide sleeve to mark the centreline of the femoral head and neck.

In both the second and third aspects, the step of moving the guide sleeve to be in line with the determined centreline may be achieved by moving a body portion that extends from a distal end to a proximal end, wherein the guide sleeve is part of the body portion and the body portion is movable so as to cause corresponding movement of the guide sleeve, so that the guide sleeve may be moved in the anterior, posterior, medial and lateral directions, so that it can be moved so as to be in line with the centreline of the femoral neck as determined by the computer device.

In both the second and third aspects, the step of moving the guide sleeve to be in line with the determined centreline may be achieved by using a guide sleeve location adjustor to move the guide sleeve to be in line with the determined centreline, wherein the targeting device comprises this guide sleeve location adjustor, suitable for moving the guide sleeve in the anterior, posterior, medial and lateral directions.

In both the second and third aspects, the targeting device may comprise a screen and the software is such that, having determined the location of the centreline of the femoral neck, it provides an image of the centreline location on the screen so that a person can manually move the guide sleeve in line with this calculated centreline position, and the step of moving the guide sleeve to be in line with the determined centreline involves moving the guide sleeve to be in line with the determined centreline as shown on the screen.

A specific embodiment of the present invention will now be described, by means of example only, with reference to the drawings, in which:

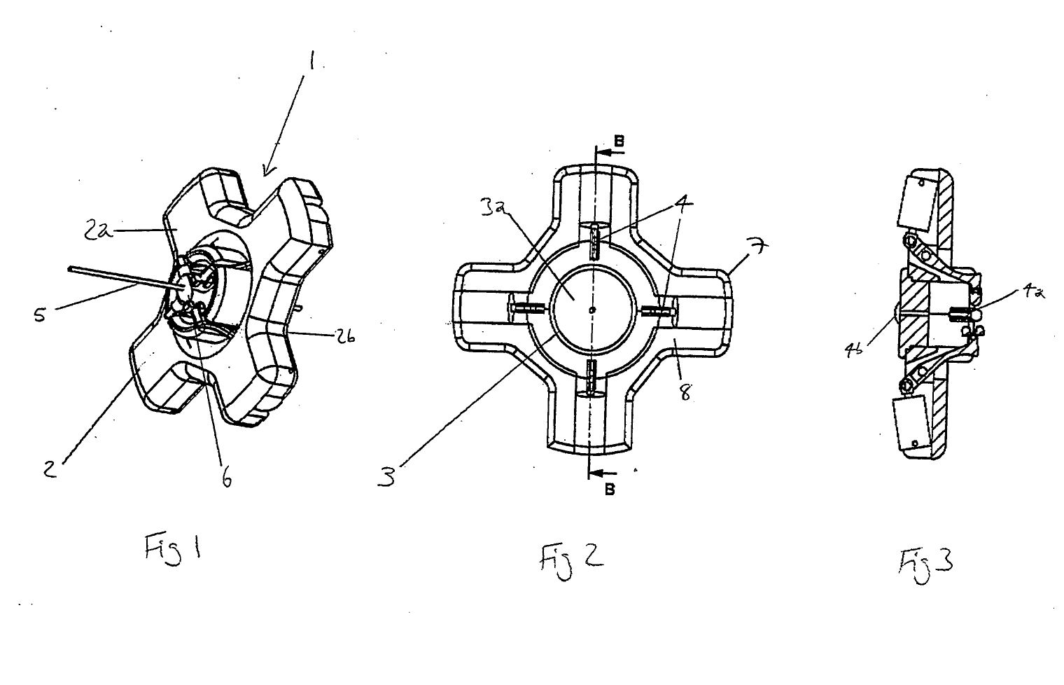

FIG. 1 shows a perspective view of a first targeting device according to the present invention;

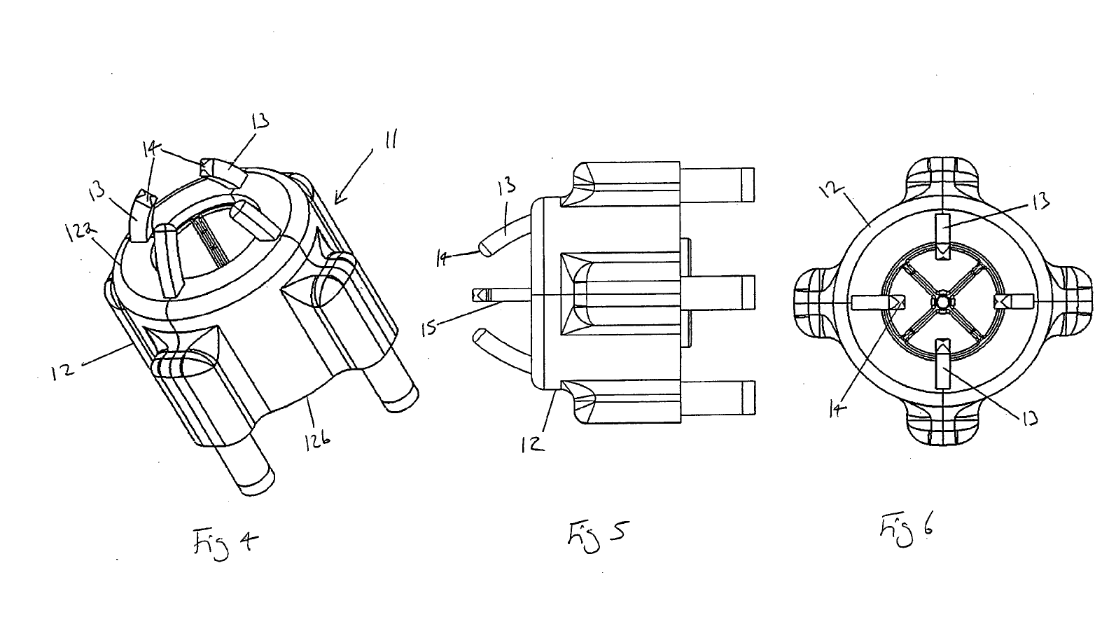

FIG. 2 shows a front view of the targeting device of FIG. 1; and

FIG. 3 shows a cross section through line B-B of the targeting device of FIG. 2;

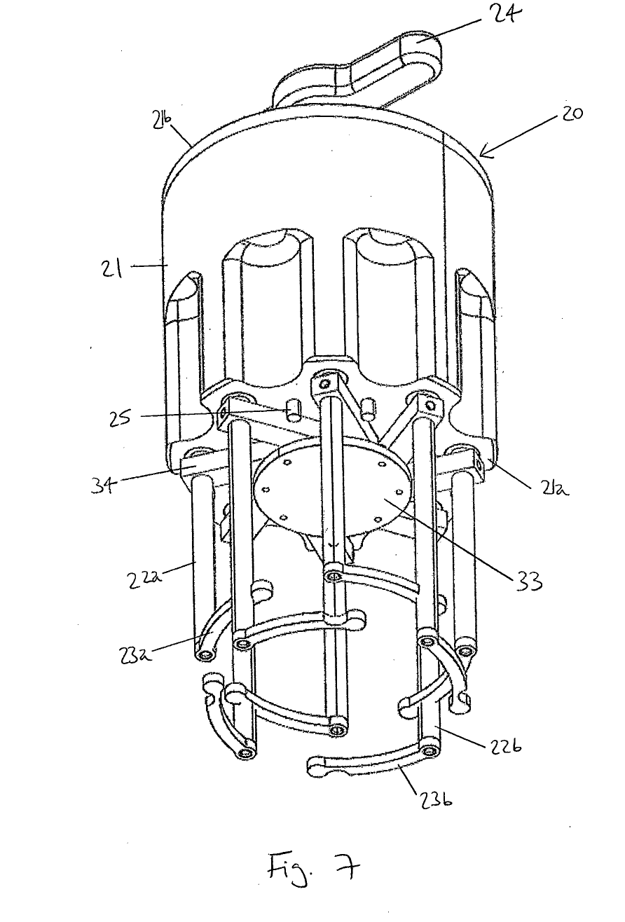

FIG. 4 shows a perspective view of a second targeting device according to the present invention;

FIG. 5 shows a side view of the targeting device of FIG. 4;

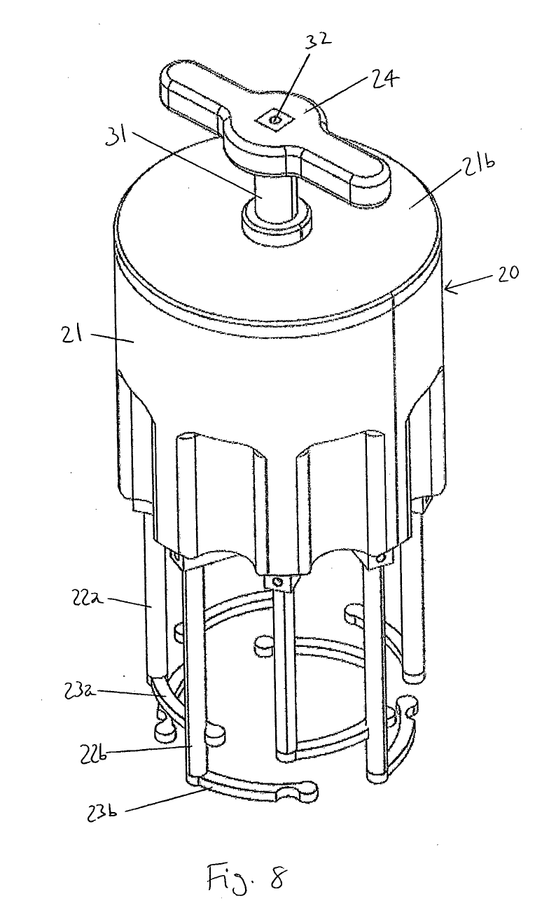

FIG. 6 shows a front view of the targeting device of FIG. 4;

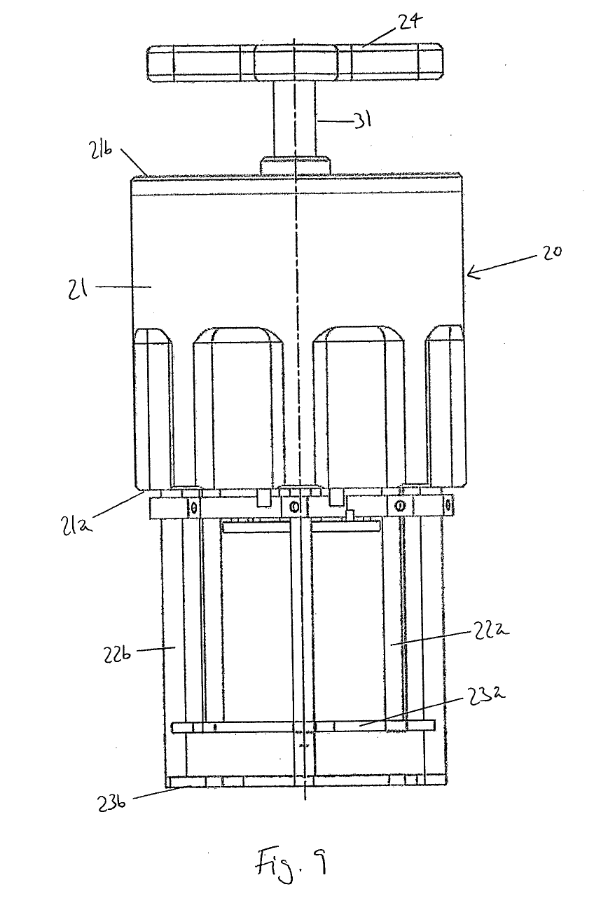

FIG. 7 shows a perspective view from below of a third targeting device according to the present invention;

FIG. 8 shows a perspective view from above of the targeting device of FIG. 7;

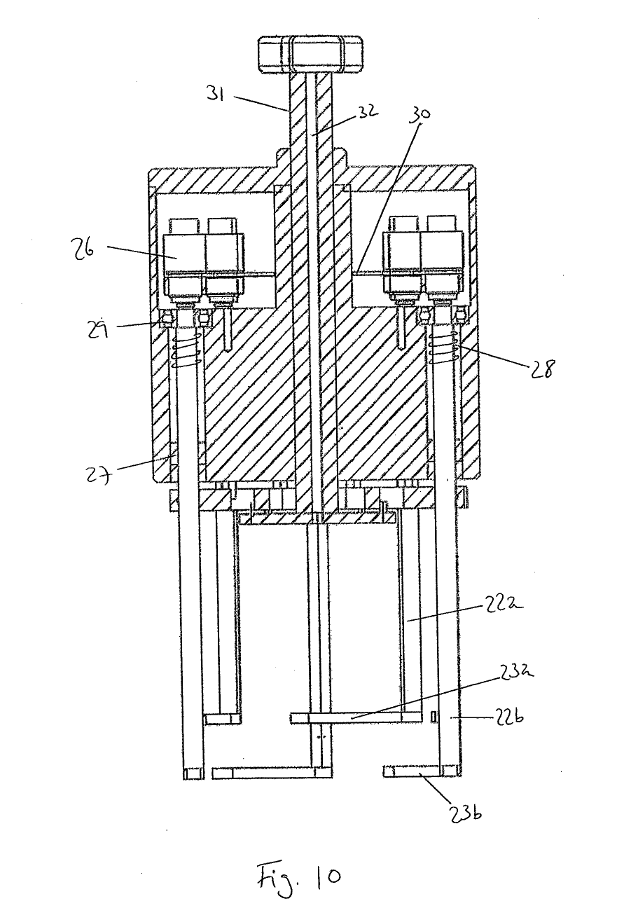

FIG. 9 shows a side view of the targeting device of FIG. 7;

FIG. 10 shows a cross section of the targeting device of FIG. 7;

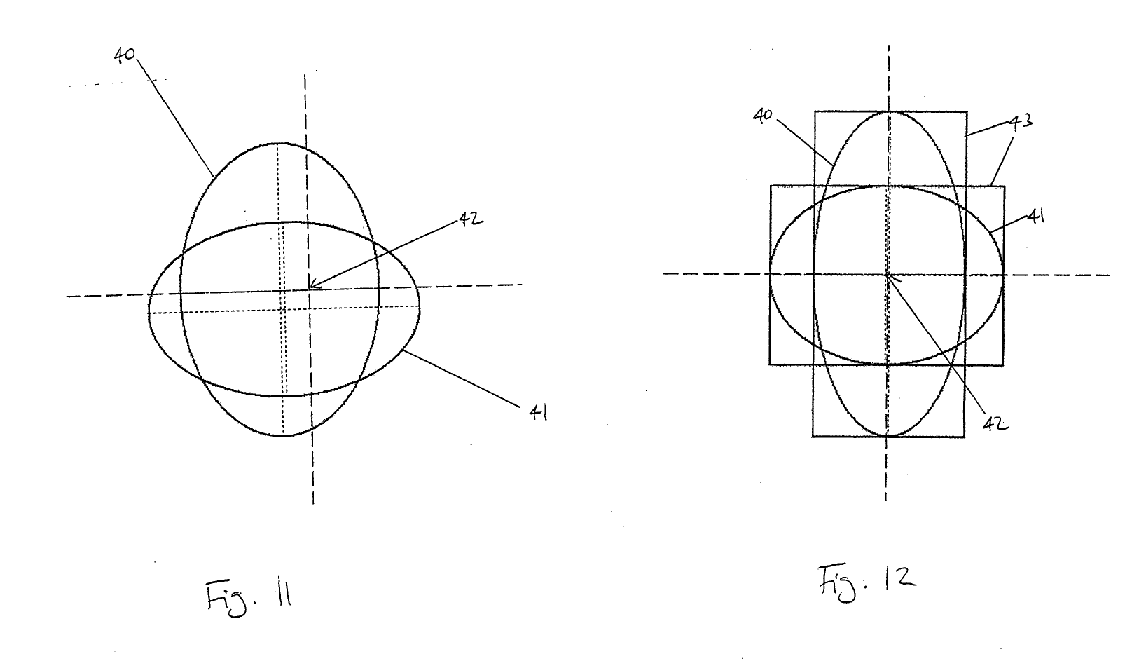

FIG. 11 shows an example of the information displayed on the screen that may be provided in the targeting device of FIG. 7, showing the device as not aligned with the calculated centreline; and

FIG. 12 shows an example of the information displayed on the screen that may be provided in the targeting device of FIG. 7, showing the device as aligned with the calculated centreline.

FIGS. 1-3 show a first targeting device 1 suitable for use in open or minimally invasive hip replacement surgery. The device 1 comprises a sleeve component 2 having a proximal end 2a and a distal end 2b. The sleeve component 2 comprises a cylindrical shaped inner wall 3. The inner wall 3 has an interior surface that defines an inner bore 3a. The sleeve component 2 is sized in view of the femoral neck in question, and can be placed over the femoral neck with some or all of the femoral neck located within the bore 3a, with the distal end 2b of the sleeve component 2 towards the base of the femoral neck.

The sleeve component also has an outer wall 7. The outer wall is in the shape of a cross. The outer wall 7 and inner wall 3 together define a cavity 8.

The device 1 also comprises four sensor devices 4 for obtaining data relating to the location of the surface of the femoral neck with respect to a reference position when some or all of the femoral neck is located within the bore of the sleeve component. Two of the devices are located to obtain data in the anterior-posterior plane and two of the devices are located to obtain data in the superior-inferior plane. The devices 4 are angularly spaced apart by 90°. The devices 4 are located within cavity 8 of the sleeve component 2.

The sensor devices 4 are electro-mechanical devices. Each electromechanical device comprises an electronically driven motor 4a with an end probe 4b that is able to move in a straight line from its neutral position to a position abutting the surface of the femoral neck. Each electromechanical device is arranged to be able to record the distance moved by the electronically driven motor and probe from the neutral position to the position abutting the surface of the femoral neck.

The device 1 further comprises a guide sleeve 5. The guide sleeve 5 is located at the proximal end 2a of the sleeve component 2. The guide sleeve is an elongate cylindrical shape with a bore capable of receiving a guide wire.

The device 1 additionally comprises guide sleeve location adjustor 6 suitable for moving the guide sleeve in the anterior, posterior, medial and lateral directions. The guide sleeve location adjustor 6 is electromechanical, comprising a combination of known motors and electronic devices to allow movement of the guide sleeve in the anterior, posterior, medial and lateral directions.

The device 1 also comprises a computer device and software (not shown) for receiving data from the sensor devices 4 and processing this data so as to determine the location of the centreline of the femoral neck. The software controls the guide sleeve location adjustor 6, causing it to move the guide sleeve in the anterior, posterior, medial and lateral directions as necessary so as to be in line with the determined centreline of the femoral neck.

In use the targeting device 1 of the present invention permits location of the central longitudinal axis of the femoral neck. In minimally invasive hip replacement surgery (as described in International Patent Publication No WO 03/065906 of the same inventor) or in open hip replacement surgery, the following steps may be carried out:
providing a targeting device 1;
placing the sleeve component 2 over the femoral neck, so that some or all of the femoral neck is located within the bore 3a of the sleeve component, with the distal end 2b located towards the base of the femoral neck, such that the sensor devices 4 are placed over the femoral neck and radially spaced therefrom;
moving each electronically driven motor 4a with an end probe 4b in a straight line from its neutral position to a position abutting the surface of the femoral neck, and recording the distance moved;
using the software to determine the position of the centreline of the femoral head and neck based on the data received from each sensor device 4;
using the guide sleeve location adjustor 6 to move the guide sleeve 5 to be in line with the determined centreline.

A guide wire can then be passed through the guide sleeve 5 to mark the centreline of the femoral neck.

The femoral neck can then be reamed accurately and centrally and the resultant prosthesis will be accurately and centrally positioned.

FIGS. 4-6 show a second targeting device 11 suitable for use in open or minimally invasive hip replacement surgery.

The device 1 comprises a main body 12 which is substantially cylindrical in shape and has a proximal end 12a and a distal end 12b.

The device 11 comprises four retractable arms 13 located at the proximal end 12a of the main body. The arms 13 are angularly spaced apart by 90°.

The arms 13 can extend from their retracted position to an extended position. The arms are curved.

The device 11 also comprises four sensor devices 14 for obtaining data relating to the location of the surface of the femoral neck with respect to a reference position when some or all of the femoral neck is located within the bore of the sleeve component. Two of the devices are located to obtain data in the anterior-posterior plane and two of the devices are located to obtain data in the superior-inferior plane.

The sensor devices 14 are located at the distal end of the retractable arms 13.

The sensor devices 14 are electro-mechanical devices. Each electromechanical device comprises an electronically driven motor with an end probe that is able to move in a straight line from its neutral position to a position abutting the surface of the femoral neck. Each electromechanical device is arranged to be able to record the distance moved by the electronically driven motor and probe from the neutral position to the position abutting the surface of the femoral neck.

The device 11 further comprises a guide sleeve 15. The guide sleeve 15 is located at the proximal end 12a of the main body 12. The guide sleeve is an elongate cylindrical shape with a bore capable of receiving a guide wire.

The device 11 additionally comprises guide sleeve location adjustor (not shown) suitable for moving the guide sleeve in the anterior, posterior, medial and lateral directions. The guide sleeve location adjustor is electromechanical, comprising a combination of known motors and electronic devices to allow movement of the guide sleeve in the anterior, posterior, medial and lateral directions.

The device 11 also comprises a computer device and software (not shown) for receiving data from the sensor devices 14 and processing this data so as to determine the location of the centreline of the femoral neck. The software controls the guide sleeve location adjustor, causing it to move the guide sleeve in the anterior, posterior, medial and lateral directions as necessary so as to be in line with the determined centreline of the femoral neck.

In use the targeting device 11 of the present invention permits location of the central longitudinal axis of the femoral neck. In minimally invasive hip replacement surgery (as described in International Patent Publication No WO 03/065906 of the same inventor) or in open hip replacement surgery, the following steps may be carried out:
providing a targeting device 11;
extending each retractable arm 13 from its retracted position to its extended position, so as to move each electromechanical sensor device 14 from a position outside the body to a position inside the body, over the femoral neck;
moving each electromechanical sensor device 14 in a straight line from its neutral position to a position abutting the surface of the femoral neck, and recording the distance moved;
using the software to determine the position of the centreline of the femoral head and neck based on the data received from each sensor device 14;
using the guide sleeve location adjustor to move the guide sleeve 15 to be in line with the determined centreline.

A guide wire can then be passed through the guide sleeve 15 to mark the centreline of the femoral neck.

The femoral neck can then be reamed accurately and centrally and the resultant prosthesis will be accurately and centrally positioned.

Although the devices 1, 11 have been illustrated in relation to the use of electromechanical sensor devices, it will be understood by the skilled man that other sensor devices, in particular radar, infra-red, ultrasound or electro-magnetic sensor devices, could equally be used.

FIGS. 7-10 show a third targeting device 20 suitable for use in open or minimally invasive hip replacement surgery. The device 20 comprises a body portion 21 having a proximal end 21a and a distal end 21b. The body portion 21 is substantially cylindrical shaped and has a hollow interior.

The device 20 also comprises eight location sensor devices for obtaining data relating to the location of the surface of the femoral neck with respect to a reference position when some or all of the femoral neck is located within the bore of the sleeve component. The location sensor devices are provided as two sets of four devices. In each set the four devices are angularly spaced apart by 90°.

Each location sensor device comprises an elongate shaft 22a, 22b, which is movably supported on the body portion 21 by ball bearings 27. The elongate shafts 22a, 22b are aligned with the elongate axis of the body portion 21. The elongate shafts 22a, 22b extend from their distal ends that are located inside the body portion 21 to their proximal ends that are located outside the body portion, via the proximal end 21a of the body portion. The elongate shafts are arranged so as to be angularly spaced around the circumference of the body portion 21.

Each location sensor device further comprises a rotating lever 23a, 23b at the proximal end of each elongate shaft 22a, 22b. The rotating lever 23a, 23b is attached to the elongate shaft 22a, 22b such that the rotational movement of the lever 23a, 23b in a plane perpendicular to the shaft 22a, 22b causes corresponding movement of the elongate shaft 22a, 22b.

Each location sensor device also comprises a rotary magnetic encoder 26 which is located at the distal end of elongate shaft 22a, 22b and is connected to the shaft 22a, 22b via a coupling bush 29, which transmits the rotation of the shaft to the encoder 26. Accordingly, the movement of the lever 23a, 23b can be measured by the encoder 26. The encoder 26 is supported on rotation support 30. The encoders 26 have a free suspension (only being supported by the coupling bush 29 and rotation support 30) to avoid misalignment problems.

Each lever 23a, 23b can move between a first, retracted, position, and a second, engaging, position. Each lever is biased towards the second position by a torsion spring 28.

The first set of location sensor devices comprises upper levers 23a and their associated shafts 22a. The second set of location sensor devices comprises lower levers 23b and their associated shafts 22b. The upper levers 23a and lower levers 23b are at different distances from the proximal end 21a of the body portion—in other words the shafts 22a are shorter than the shafts 22b. Accordingly, the upper levers 23a and the lower levers 23b will contact the femoral neck surface at different distances from the femoral head.

The targeting device 20 further comprises a wheel 33, located at the proximal end of the body portion 21. The wheel 33 is provided with a handle 24 that is turned by the user in order to rotate the wheel. The wheel 33 is linked to the handle 24 via central shaft 31.

Each of the shafts 22a, 22b has an arm 34 fixedly extending therefrom, at or near to the junction of the shaft with the proximal end 21a of the body portion. Each arm 34 extends towards the central elongate axis of the body portion 21.

The wheel 33 is provided with pins 25 that abut each of these arms 34.

Accordingly, the turning of the wheel 33 by its handle 24 will move the pins 25, which will then push the arms 34 and this will in turn move the shafts 22a, 22b, which will correspondingly move the levers 23a, 23b towards their first position. Accordingly, the wheel 33 can be rotated by the user, to counteract the force of the spring 28 and move the levers 23a, 23b from their second position, towards which they are biased, towards their first positions. This can be used to move the levers 23a, 23b out of the way to facilitate movement of the levers over the femoral head to reach the desired location over the femoral neck.

The movement of each lever 23a, 23b is also limited by the stopper pins 25 mounted on the proximal end of the body portion 21 of the targeting device.

The device may be also provided with a securing system (not shown), which enables the user to secure the levers 23a, 23b in the first position.

The device may be also provided with a securing system (not shown), which enables the user to secure the levers 23a, 23b in the second position.

The device 20 further comprises a guide sleeve 32. The guide sleeve 32 runs through the central elongate axis of the body portion 21, from the distal end 21b to the proximal end 21a. The guide sleeve is an elongate cylindrical shape with a bore capable of receiving a guide wire.

The device 20 also comprises a computer device and software (not shown) for receiving data from the encoders 26 and processing this data so as to determine the location of the centreline of the femoral neck. The software provides information about the centreline of the femoral neck and the location of the centreline of the guide sleeve on a screen.

The device 20 also includes a screen. The screen is not shown in FIGS. 7 to 10, but an example of the information that may be shown on the screen is given in FIGS. 11 and 12.

In FIG. 11, data from the encoders associated with the upper levers 23a and their associated shafts 22a is shown as ellipse 40. This is drawn from data points as to the location of the outer surface of the femoral neck that have been calculated from the degree of rotation of each of the four levers 23a that was required for them to come into contact with the femoral neck surface.

Data from the encoders associated with the lower levers 23b and their associated shafts 22b is shown as ellipse 41. This is drawn from data points as to the location of the outer surface of the femoral neck that have been calculated from the degree of rotation of each of the four levers 23b that was required for them to come into contact with the femoral neck surface.

Each ellipse is therefore an approximation as to the outer surface shape of the femoral neck at a given distance along the femoral neck from the femoral head.

The screen may also display the centre of each of the ellipses 40 and 41, as calculated from the data points.

The screen also displays the location of the centreline of the guide sleeve, which is presented on the screen by cross hatched lines that meet at centreline 42.

Accordingly, a person can manually move the body portion 21, and hence the guide sleeve 32, until the location of the centreline of the guide sleeve 42 is lined up with the centre of the ellipses 40 and 41.

When the centreline of the guide sleeve 42 is lined up with the centre of the ellipses 40 and 41, this may be indicated to the user by the appearance of rectangular boxes 43 on screen, as shown in FIG. 12, or by some other sort of message or image on screen.

In use the targeting device 20 of the present invention permits location of the central longitudinal axis of the femoral neck. In minimally invasive hip replacement surgery (as described in International Patent Publication No WO 03/065906 of the same inventor) or in open hip replacement surgery, the following steps may be carried out:
providing a targeting device 20, with the levers 23a, 23b in their first, retracted, positions;
placing the shafts 22a, 22b and levers 23a, 23b over the femoral neck;
allowing the spring 28 to bias the levers 23a, 23b to their second positions, with the degree of rotation to achieve this being recorded by the encoders 26;
using the software to determine the position of the centreline of the femoral head and neck based on the data received from each encoder 26;
moving the guide sleeve 32 to be in line with the determined centreline.

A guide wire can then be passed through the guide sleeve 32 to mark the centreline of the femoral neck.

The femoral neck can then be reamed accurately and centrally and the resultant prosthesis will be accurately and centrally positioned.

1. A targeting device for use in hip surgery to allow the position of the centreline of a femoral neck to be located in a body, the device comprising:
at least one location sensor device, wherein at least part of each location sensor device is movable to a position inside the body that is over the femoral neck, the at least one sensor device being suitable for obtaining data relating to a location of a surface of the femoral neck with respect to a reference position, in both the anterior-posterior and superior-inferior planes;
a guide sleeve, having a bore capable of receiving a guide wire; and
a computer device with software for receiving data from the at least one location sensor device and processing the data so as to determine the location of the centreline of the femoral neck;
wherein the guide sleeve can be moved in the anterior, posterior, medial and lateral directions, so as to be in line with the centreline of the femoral neck as determined by the computer device.

2. The targeting device of claim 1, wherein the at least one location sensor device is electromechanical.

3. The targeting device of claim 1, wherein the part of each location sensor device that is movable to a position inside the body that is over the femoral neck is positioned inside the body over the femoral neck and radially spaced therefrom.

4. The targeting device of claim 1, wherein the at least one location sensor device comprises at least two location sensor devices, with at least one of the location sensor devices operable to obtain data in the anterior-posterior plane and at least another one of the location sensor devices operable to obtain data in the superior-inferior plane.

5. The targeting device of claim 4, wherein the at least one sensor device comprises at least four location sensor devices.

6. The targeting device of claim 5, wherein at least two of the location sensor devices are operable to obtain data in the anterior-posterior plane and at least two of the location sensor devices are operable to obtain data in the superior-inferior plane.

7. The targeting device of claim 1, wherein the at least one location sensor device comprises at least two sets of location sensor devices, wherein each set includes one or more location sensor devices, wherein at least a first part of each location sensor device is movable to a position inside the body that is over the femoral neck and wherein a second part of each location sensor device is movable so as to be in contact with the femoral neck surface, wherein each location sensor device includes a sensor for obtaining information about the location of the second part that is movable so as to be in contact with the femoral neck surface, such that each set of location sensor devices is suitable for obtaining data relating to the location of the surface of the femoral neck with respect to a reference position, in both the anterior-posterior and superior-inferior planes.

8. The targeting device of claim 7, wherein the sets of location sensor devices are spaced apart by a distance of 5 mm or more.

9. The targeting device of claim 8, wherein the sets of location sensor devices are spaced apart by a distance of 10 mm or more.

10. The targeting device of claim 7, wherein each set of location sensor devices comprises at least four location sensor devices.

11. The targeting device of claim 10, wherein each set of location sensor devices has four angularly spaced location sensor devices, spaced at angles of 90° apart.

12. The targeting device of claim 1, wherein a second part of each location sensor device is movable so as to be in contact with the femoral neck surface, wherein each location sensor device includes a sensor for obtaining information about the location of the second part that is movable so as to be in contact with the femoral neck surface, such that the at least one location sensor device is suitable for obtaining data relating to the location of the surface of the femoral neck with respect to a reference position, in both the anterior-posterior and superior-inferior planes.

13. The targeting device of claim 12, wherein the targeting device further comprises a body portion that extends from a distal end to a proximal end, wherein the guide sleeve is part of the body portion and the body portion is movable so as to cause corresponding movement of the guide sleeve, so that the guide sleeve may be moved in the anterior, posterior, medial and lateral directions, so that the guide sleeve can be moved to be in line with the centreline of the femoral neck as determined by the computer device.

14. The targeting device of claim 13, wherein at least part of each location sensor device is located within the body portion, and at least part of each location sensor device extends from the proximal end of the body portion.

15. The targeting device of claim 12, wherein the second part of each location device that is movable so as to be in contact with the femoral neck surface comprises a lever which has one fixed end and one movable end and pivots about its fixed end, with the movable end of the lever moving between a first, retracted, position and a second, engaging, position.

16. The targeting device of claim 15 wherein the lever is biased towards the second position.

17. The targeting device of claim 16, wherein movement of the lever from the first position to the second position is caused by a spring or other elastic device that exerts a biasing force.

18. The targeting device of claim 15, wherein the lever is mounted at its fixed end on an elongate shaft that extends from a distal end to a proximal end, wherein movement of the lever causes corresponding movement of the shaft.

19. The targeting device of claim 18, wherein the lever is a rotating lever.

20. The targeting device of claim 15, wherein the lever can be moved from the second position, towards which it is biased, towards the first position, by a manual location adjustor.

21. The targeting device of claim 20, wherein the targeting device is provided with a securing system, which enables a user of the targeting device to secure the levers in the first position.

22. The targeting device of claim 15, wherein the sensor for obtaining information about the location of the part that is movable so as to be in contact with the femoral neck surface comprises a rotating lever and the sensor for obtaining information about the location of the lever is a sensor selected from the group consisting of a rotary encoder or a potentiometer.

23. The targeting device of claim 22, wherein the sensor comprises a magnetic rotary shaft encoder.

24. The targeting device of claim 12, wherein the targeting device further comprises a screen and the software is such that, having determined the location of the centreline of the femoral neck, it provides information regarding the centreline location on the screen so that a user can manually move the guide sleeve in line with a calculated centreline position.

25. The targeting device of claim 1, wherein the at least one location sensor device is movable to a position inside the body that is radially spaced from the femoral neck, and wherein the targeting device further comprises:
a guide sleeve location adjustor, suitable for moving the guide sleeve in the anterior, posterior, medial and lateral directions.

26. The targeting device of claim 1, wherein portions of the targeting device that are intended to enter into the body are made from material that can be steam sterilized.

27. A method of locating the centreline of the osteotomised base of the femoral neck at the head-neck junction, which method comprises:
providing a targeting device for use in hip surgery to allow the position of a centreline of the osteotomised base of the femoral neck at the head-neck junction to be located in a body, the device comprising:
at least one location sensor device, wherein at least part of each location sensor device is movable to a position inside the body that is over the femoral neck, the at least one sensor device being suitable for obtaining data relating to a location of a surface of the femoral neck with respect to a reference position, in both the anterior-posterior and superior-inferior planes;
a guide sleeve, having a bore capable of receiving a guide wire; and
a computer device with software for receiving data from the at least one location sensor device and processing the data so as to determine the location of the centreline of the femoral neck;
wherein the guide sleeve can be moved in the anterior, posterior, medial and lateral directions, so as to be in line with the centreline of the femoral neck as determined by the computer device;
moving at least part of the at least one location sensor device to a position inside the body, over the femoral neck;
using the at least one location sensor device to obtain data relating to the location of the surface of the femoral neck with respect to a reference position in at least one location in the anterior-posterior plane and at least one location in the superior-inferior plane;
using the software to determine the position of the centreline of the femoral neck based on the data received from the at least one location sensor device; and
moving the guide sleeve to be in line with the determined centreline.

28. A method of locating the centreline through the femoral neck and femoral head, which method comprises:
providing a targeting device for use in hip surgery to allow the position of a centreline of through the femoral neck and the femoral neck to be located in a body, the device comprising:
at least one location sensor device, wherein at least part of each location sensor device is movable to a position inside the body that is over the femoral neck, the at least one sensor device being suitable for obtaining data relating to a location of a surface of the femoral neck with respect to a reference position, in both the anterior-posterior and superior inferior planes;
a guide sleeve, having a bore capable of receiving a guide wire; and
a computer device with software for receiving data from the at least one location sensor device and processing, the data so as to determine the location of the centreline of the femoral neck;
wherein the guide sleeve can be moved in the anterior, posterior, medial and lateral directions, so as to be in line with the centreline of the femoral neck as determined by the computer device;
moving at least part of the at least one location sensor device to a position inside the body, over the femoral neck;
using the at least one location sensor device to obtain data relating to the location of the surface of the femoral neck with respect to a reference position in at least one location in the anterior-posterior plane and at least one location in the superior-inferior plane;
using the software to determine the position of the centreline of the femoral head and femoral neck based on the data received from the at least one location sensor device; and
moving the guide sleeve to be in line with the determined centreline.
